(19) United States
(12) Patent Application Publication
(10) Pub. No.: US 2026/0215835 A1
(43) Pub. Date: Jul. 30, 2026

(54) SUPPLY APPARATUS AND METHOD FOR SUPPLYING AN ELECTROMEDICAL INSTRUMENT

(71) Applicant: Erbe Elektromedizin GmbH, Tuebingen (DE)

(72) Inventors: Rolf WEILER, Tuebingen (DE); Philipp MAIER, Tuebingen (DE)

(21) Appl. No.: 19/455,147

(22) Filed: Jan. 21, 2026

(30) Foreign Application Priority Data
Jan. 28, 2025 (EP) 25154540.6

(51) Int. Cl. A61B 18/12; A61B 18/00

(52) U.S. Cl. CPC A61B 18/1206; A61B 2018/00642; A61B 2018/0072; A61B 2018/00767; A61B 2018/00875

(57) ABSTRACT

Supply apparatuses and methods for supplying an electromedical instrument with electrical energy are disclosed. For example, an adjustable and/or selectable operating mode parameter (M) defines an operating mode voltage (U_{M}) that indicates a set point value for an electrode voltage (U_{E}) at an electrode of the electromedical instrument. At a power supply output, an impressed output voltage (U_{A}) is provided in accordance with a set point output voltage (U_{S}) as well as a variable output current (I_{A}) for the electromedical instrument. In one as aspect, an operating mode voltage is corrected based on a correction value and/or voltage and forms a corrected operating mode voltage (U_{MC}) based on which the set point output voltage (U_{S}) is provided for the power supply. The correction value or voltage are determined depending based on an actual value of the output current (I_{A}) as well as an impedance parameter (ZI) that characterizes the impedance of the instrument.

This application claims priority to European Patent Application No. 25154540.6. filed Jan. 28, 2025, the entirety of which is incorporated herein.

The invention relates to a supply apparatus for an electromedical instrument, particularly an electrosurgical instrument. The supply apparatus is configured to supply an electromedical instrument connected thereto with electrical energy, particularly in order to provide electrical power for an electrode of the instrument. The invention also relates to a method for supplying an electromedical instrument with electrical energy as well as a system comprising a supply apparatus and an electromedical instrument connected thereto.

DE 10 2015 212 359 A1 discloses a high-frequency electrosurgical instrument that can be electrically connected to a high-frequency generator by means of a connector and a cable. The high-frequency generator can be configured to switch off the electrical supply for the instrument if a short circuit between two electrodes of the instrument has been determined. In order to not affect the short circuit determination by the resistance of the cable or the electrosurgical instrument, the instrument comprises an adaption circuit for impedance adaption. By means of the adaption circuit parasitic capacitances and/or inductances of the cable and the instrument shall be compensated. The adaption circuit can also comprise a tuning inductor in order to tune a resonance frequency of the high-frequency electrosurgical instrument to a desired operating frequency of the high-frequency generator.

However, such an adaption circuit must be provided for each instrument and, if required, for each type of application of an instrument. This makes instruments expensive and complex, which is particularly undesired for disposable instruments.

DE 689 118 67 T2 relates to a method and a supply apparatus for supplying a patient electrode with electrical power. A power amplifier, to which the patient electrode is connected, creates output measurement signals corresponding to the output voltage and the output current. From these measurements the current load impedance at the output of the power amplifier is calculated and depending therefrom, the electrical power that has to be provided at the output of the power amplifier is determined by means of a curve or set of curves and is controlled in a feedback manner accordingly. If the impedance, however, is affected due to parasitic effects or similar, this can result in an incorrect assignment of a power value to be set.

Different electromedical instruments and different applications of an electromedical instrument can result in that an impedance at the output of a supply apparatus changes to which the electromedical instrument is connected. The impedance that changes depending on the instrument and/or its operating mode can in turn result in that an electrode of the electromedical instrument is not provided with the setpoint voltage set in an open- or closed-loop control of the supply apparatus and thus undesired voltage deviations occur. Such voltage deviations can be disadvantageous during treatment of biological tissue.

It is therefore the object of the present invention to reduce or avoid undesired voltage deviations in an electromedical instrument, in particular at least one electrode serving for treating tissue, when supplying it with electrical power or energy.

This object is solved, for example, by means of a supply apparatus having the features of claim 1, a system having the features of claim 10, as well as a method having the features of claim 14.

The supply apparatus according to the invention is configured for supply of an electromedical instrument with electrical power or energy. The supply apparatus comprises a power supply providing an impressed output voltage at its power supply output. The output voltage is preferably controlled in a feedback manner by means of a power supply controller according to a preset setpoint output voltage. At the power supply output the power supply provides an output current that can vary depending on the load.

The impedance of the electromedical instrument connected to the supply apparatus can vary. For example, electromedical instruments of different type have different impedances. In addition, even electromedical instruments of similar type can have different impedances due to components that are subject to tolerances. Such tolerances and fluctuations of the impedance result in deviations of a voltage applied for the treatment of biological tissue, particularly an electrode of the instrument configured for treatment of biological tissue. The electrode can be arranged on a distal end of the instrument. For example, a voltage deviation can be due to a reactive voltage component caused by a not impressed output current and the impedance. Then, in the instrument, for example at the electrode of the instrument via which the apparatus and the biological tissue are connected with each other, the desired voltage (for example electrode voltage) is no longer available.

To avoid this, the supply apparatus comprises a setpoint adjustment device. The setpoint adjustment device is configured to determine the setpoint output voltage so that a reactive voltage component caused by the impedance of the electromedical instrument is partly or entirely compensated.

The setpoint adjustment device comprises a determination unit as well as a correction unit for this purpose. The correction unit is configured to determine a correction value depending on a current actual value of an output current at the output of the power supply apparatus and depending on at least one impedance parameter. The impedance parameter characterizes the electrical impedance of the electromedical instrument to be supplied. The impedance parameter can be considered as internal resistance of a voltage source. For example, the impedance parameter can be determined and stored for an instrument type or for each electromedical instrument individually by means of simulation and/or measurement. The impedance parameter can either be stored in a memory of the electrosurgical instrument and/or in a memory of the supply apparatus and/or in an external memory (Cloud memory). However, the impedance parameter can be assigned to an instrument type or an individual electromedical instrument and is available for the correction unit.

At least one impedance value can be used as the at least one impedance parameter. Additionally or alternatively, the impedance parameter can also be an equivalent circuit or an equivalent circuit diagram (for example complex equivalent circuit diagram with real and imaginary part), a function, a characteristic curve, a characteristic set of curves or the like. The at least one impedance parameter can in turn be dependent on at least one additional parameter, such as frequency of the output voltage and/or the output current.

The correction value is used to correct a preset operating mode voltage for a present operating mode of the supply apparatus and to therefrom determine the set point output voltage, which is then provided to the power supply. Thus, the power supply creates an output voltage that is corrected depending on the impedance parameter of the electromedical instrument to be supplied. In doing so, in the instrument, particularly at an electrode of the instrument, the desired instrument voltage or electrode voltage can be applied with only little or no deviation. A voltage applied to the impedance in the instrument and thus a voltage depending on the impedance parameter does not have to be measured thereby.

Particularly, an operating mode parameter can be selected and set by means of an operating interface or another device of the supply apparatus. The operating mode parameter defines the operating mode voltage or is the operating mode voltage that shall be provided for the selected operating mode of the supply apparatus as instrument voltage or electrode voltage. The correction value C defines a correction voltage or is a correction voltage, wherein the operating mode voltage is corrected based on the correction voltage by means of the set point adjustment device, so that a corrected operating mode voltage is formed, the absolute value of which is particularly greater than the absolute value of the operating mode voltage. In a preferred embodiment the corrected operating mode voltage can be the sum of the operating mode voltage and the correction voltage. Alternatively, the operating mode voltage can also be corrected using a correction factor which in this case represents the correction value.

A correction value and, for example, the correction voltage can be determined based on a function or an algorithm that uses the present value of the output current and the impedance parameter as input parameters.

By determining the impedance parameter of the electromedical instrument and detecting the output current during operation and providing it to the correction unit, the operating mode voltage selected via the operating mode parameter can always be adapted during operation of the supply apparatus, that is during use of an electromedical instrument connected thereto, so that in the electromedical instrument the desired instrument voltage or electrode voltage is provided.

In an embodiment, the impedance parameter can be stored in the correction unit for one or more electromedical instruments that can be connected to the supply apparatus, wherein the impedance parameter can be variable or non-variable.

In an embodiment of the supply apparatus, it is configured to determine a characteristic value of an electromedical instrument connected to the supply apparatus and, for example, to read it out of a memory of the electromedical instrument. The characteristic value can characterize the individual electromedical instrument and/or the instrument type of the electromedical instrument. Based on the characteristic value in the supply apparatus and particularly by means of the correction unit, the impedance parameter that belongs to the connected electromedical instrument can be determined in the supply apparatus and particularly by means of the correction unit. In the simplest case, the characteristic value can already comprise the impedance parameter or the characteristic value is the impedance parameter.

It can be advantageous to test the electromedical instruments after production and to determine their individual impedance and to store it in a memory of the electromedical instrument, so that the impedance parameter can be read out by the supply apparatus. Alternatively, it can also be expedient to assign an impedance parameter to a characteristic value of the connected electromedical instrument provided to the supply apparatus by means of a table or another assignment rule by means of the correction unit.

An electromedical instrument can comprise one single impedance parameter or multiple impedance parameters. For example, different types of use or different operating modes of an electromedical instrument can be assigned an impedance parameter in each case. For example, an electromedical instrument can have a different impedance parameter in a cutting mode than in a coagulation mode. It is therefore advantageous if the impedance parameter is determined depending on the present type of operation or the present operating mode. The operating mode can be determined based on the operating mode parameter, for example.

Any embodiment of the supply apparatus as explained above can be part of a system that additionally comprises an electromedical instrument connected to the supply apparatus. The electromedical instrument is preferably an electrosurgical instrument.

In different embodiments the electromedical instrument can be a monopolar instrument or a bipolar instrument. In the case of a bipolar instrument the electrode voltage is preferably determined between two electrodes of the instrument. In the case of a monopolar instrument a separate neutral electrode can be provided, which is configured for being attached to the patient. The electrode voltage can then be determined between the electrode of the monopolar instrument and the separate neutral electrode. In both cases, the operating mode voltage defined by means of the operating mode parameter is a set point value for the electrode voltage. Accordingly, the instrument can have at least one, two or also more than two electrodes, for example if the electromedical instrument is configured for different types of use, such as for cutting of tissue as well as for coagulation of tissue.

Preferably, the electromedical instrument has an instrument circuit, wherein the instrument circuit comprises at least one electrical and/or electronic component. The instrument circuit comprises particularly a capacitive component and/or an inductive component. For example, the instrument circuit can comprise a coupling capacitor to which the electrode or one of the provided electrodes is connected, preferably directly. In addition, or alternatively, the instrument circuit can also have a transformer, for example an autotransformer, and/or controllable components and/or switchable components.

A method according to the invention can be carried out using any embodiment of a supply apparatus or a system as described above.

The method according to the invention is configured to supply an electromedical instrument. First, a load-dependent variable output current as well as an impressed output voltage are provided. For example, the impressed output voltage can be controlled in closed-loop manner by means of a power supply controller according to a preset setpoint output voltage.

Depending on an impedance parameter of the electromedical instrument and depending on a present value of the output current, a correction value is determined, preferably using a function or an algorithm. The correction value is used together with an operating mode parameter in order to therefrom determine a setpoint output voltage for the power supply. Particularly, the operating mode parameter can define an operating mode voltage that in turn represents the setpoint value of an instrument voltage or electrode voltage in the electromedical instrument to be supplied. In order to reduce or eliminate deviations of the instrument or electrode voltage from the operating mode voltage, the correction value is determined depending on the impedance and particularly also depending on the output current and is used for correction of the operating mode voltage defined by the operating mode parameter.

Advantageous embodiments of the invention are derived from the dependent claims, the description and the drawings. In the following, preferred embodiments of the invention are explained in detail based on the attached drawings. The drawings show:

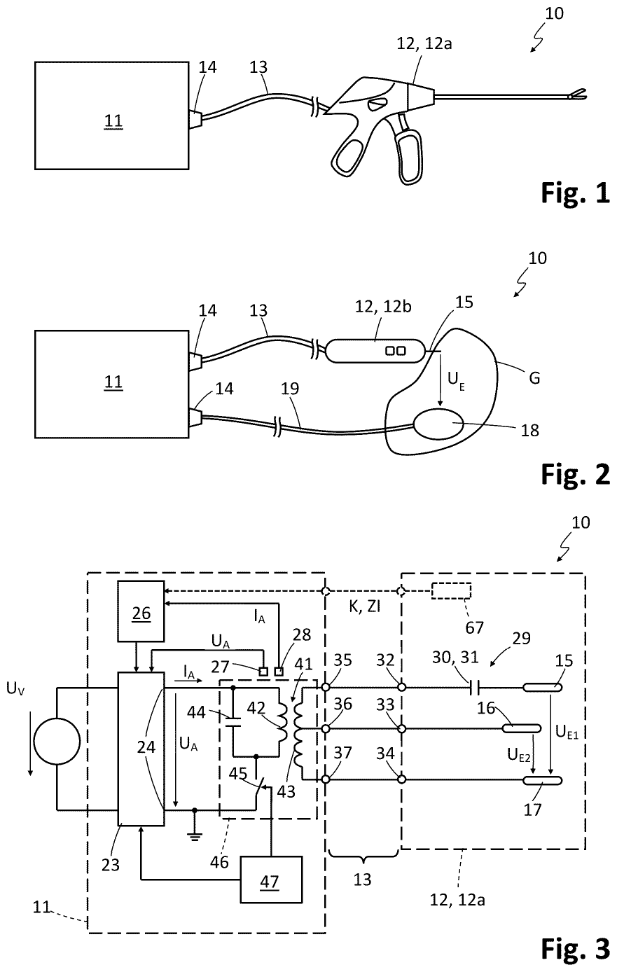

FIG. 1 shows a schematic view of an embodiment of a system comprising a supply apparatus and a bipolar electromedical instrument connected thereto,

FIG. 2 shows a schematic view of an embodiment of a system comprising a supply apparatus and a monopolar electromedical instrument connected thereto and

FIG. 3 shows a block diagram of an embodiment of a supply apparatus as well as an electromedical instrument,

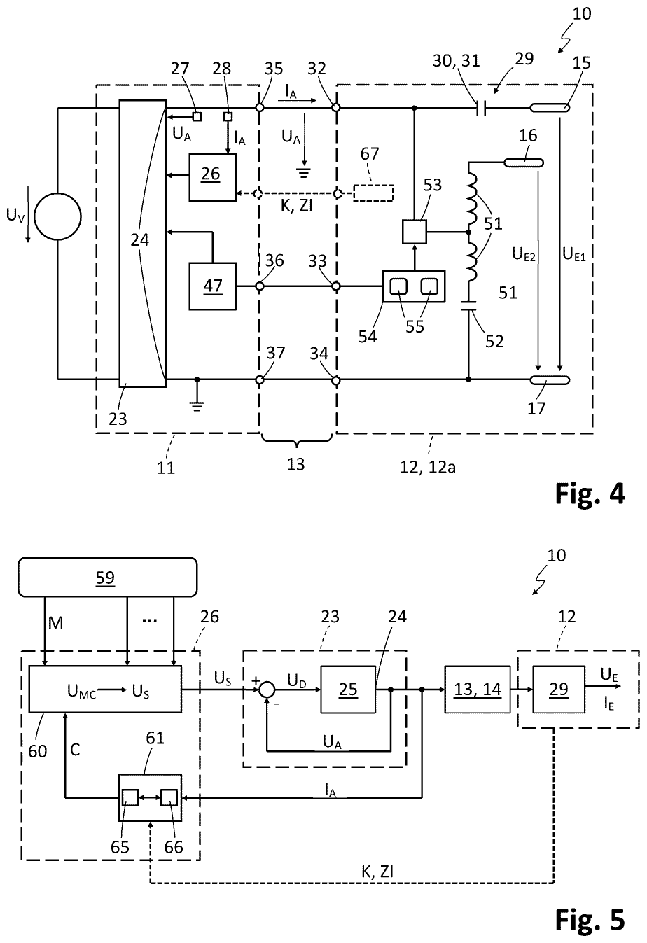

FIG. 4 shows a block diagram of another embodiment of a supply apparatus and an electromedical instrument,

FIG. 5 shows a block diagram of an embodiment for determination of a set point output voltage in the present invention and according to the present invention,

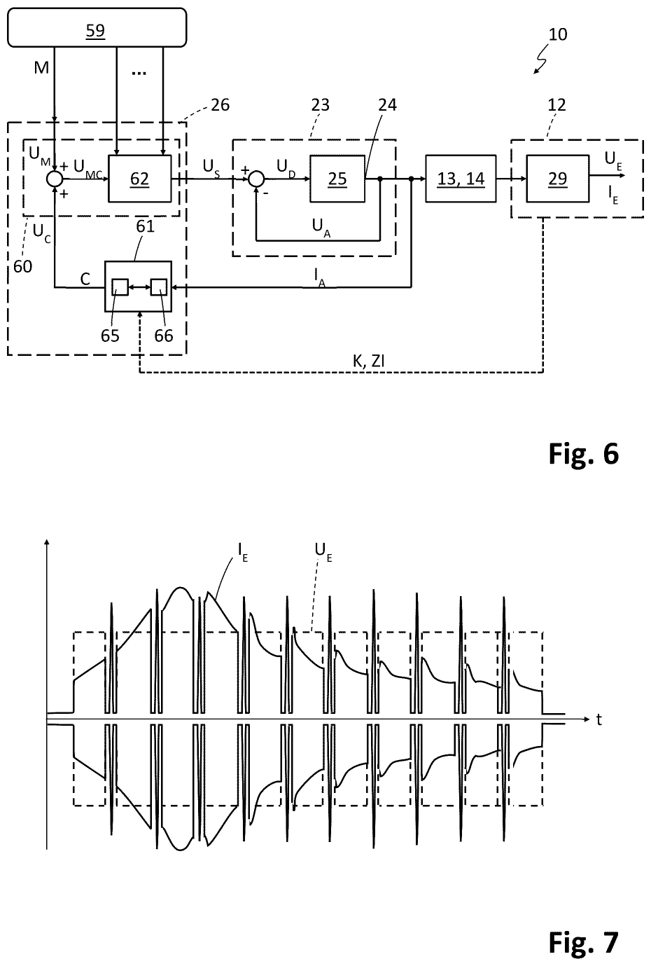

FIG. 6 shows a block diagram of an embodiment for determination of a set point output voltage in the present invention and

FIG. 7 shows an exemplary progress of a current and an electrode voltage at an electrode of an electromedical instrument during use of the present invention.

FIGS. 1 and 2 show schematically different embodiments of a system 10. The system 10 comprises a supply apparatus 11 as well as an electromedical instrument 12 that is connected or can be connected to the supply apparatus 11. The electromedical instrument 12 can be connected to the supply apparatus 11 using a cable 13 and a connector 14. When connected, supply apparatus 11 supplies the electromedical instrument 12 with electrical energy or electrical power. As an option also other media for operation of the electromedical instrument 12 can be provided by means of supply apparatus 11, such as a liquid or a gas.

In the embodiment illustrated in FIGS. 1 and 2, the electromedical instrument 12 is an electrosurgical instrument for coagulation and/or cutting of tissue G to be treated, which is subsequently briefly denoted as instrument 12.

According to the example, the instrument 12 can be configured as a bipolar instrument 12a (FIG. 1) or as a monopolar instrument 12b (FIG. 2). The bipolar instrument 12a has two or more electrodes and in the embodiment a first electrode 15, a second electrode 16, as well as a third electrode 17. The first electrode 15 can be used for coagulation of tissue in cooperation with third electrode 17. The second electrode 16 can cooperate with third electrode 17, for example, to cut tissue. Other applications and a different number of electrodes can be selected depending on the type of the instrument 12.

The monopolar instrument 12b has only one single electrode 15. While in the bipolar instrument 12a a treatment circuit can be closed via the first electrode 15 or the second electrode 16 through the tissue to be treated and further via the third electrode 17 back to the supply apparatus 11. An additional neutral electrode 18 is provided for the monopolar instrument 12b that can be connected with the supply apparatus 11 via an electrode cable 19 and a connector 14. The neutral electrode 18 is attached to the patient in electrically conductive connection with the tissue G to be treated, as highly schematically shown in FIG. 2. Typically, the neutral electrode 18 is attached on the outside of the patient's skin.

An instrument having multiple poles (bipolar or a multipolar instrument) can optionally—analog to the monopolar instrument—also be operated in a monopolar operating mode with one of the provided electrodes. In this operating mode then, additionally, the neutral electrode 18 is used.

In the following, embodiments of the system 10 or the electromedical instrument 12 are explained based on a bipolar instrument 12a. These explanations can be transferred in analog manner to a monopolar instrument 12b with one single electrode 15 or a monopolar operating mode used in a bi- or multipolar instrument 12b, wherein the function of the reference electrode is not performed by third electrode 17, but neutral electrode 18.

An embodiment of a system 10 in form of a circuit diagram or block diagram is illustrated in FIG. 3. The supply apparatus 11 is connectable to an energy supply source providing a supply voltage U_{V}. The supply voltage U_{V} is particularly the grid voltage of a power supply grid. The supply voltage U_{V} is provided to a power supply 23 of supply apparatus 11, which is configured to provide a variable output current I_{A} and an impressed output voltage U_{A} at the voltage supply output 24. The output voltage U_{A} can be controlled, for example, by means of a power supply controller 25 (FIGS. 5 and 6) in a closed control loop, so that it corresponds to a preset set point output voltage U_{S}.

For determination of the set point output voltage U_{S}, supply apparatus 11 comprises a set point adjustment device 26 providing the determined set point output voltage U_{S} for the power supply 23.

The output voltage U_{A} is detected by means of a voltage sensor 27 and is provided to the power supply 23. By means of a current sensor 28 the output current I_{A} is detected and provided to the set point adjustment device 26. The set point adjustment device 26 is configured to determine and to provide to the power supply 23 the set point output voltage U_{S} depending on the present value of the output current I_{A}.

In addition, an impedance parameter ZI is provided to the set point adjustment device 26 or the impedance parameter ZI is determined depending on another parameter, particularly a characteristic value K. The impedance parameter ZI characterizes the impedance of the instrument 12 that is to be supplied or that is connected to supply apparatus 11. Particularly, the impedance is defined by an instrument circuit 29 of instrument 12, that comprises at least one capacitive and/or inductive component 30. In the embodiment illustrated in FIG. 3 instrument circuit 29 has only one single component 30, namely a coupling capacitor 31. One of the electrodes is connected to the coupling capacitor 31, which is presently shown as being connected to the first electrode 15. In modification to the embodiment according to FIG. 3, instrument circuit 29 can comprise other or additional electrical and/or electronic components, for example a transformer, semi-conductor switches, switches or push buttons operable by an operator, etc. The instrument circuit 29 according to FIG. 3 therefore represents a very simple realization having a coupling capacitor 31 only.

In the embodiment illustrated in FIG. 3, first electrode 15 is electrically connected with a first instrument input 32 via coupling capacitor 31. The second electrode 16 is electrically connected with a second instrument input 33 and third electrode 17 is electrically connected with a third instrument input 34. The instrument inputs 32, 33, 34 are electrically connected with a respectively assigned apparatus output via cable 13, according to the example with a first apparatus output 35, a second apparatus output 36 and a third apparatus output 37 of supply apparatus 11.

The instrument voltage applied in the instrument 12 is the electrode voltage U_{E} in the embodiments. The electrode voltage U_{E} is determined between an active electrode (for example first electrode 15 and/or second electrode 16) on one hand and a reference electrode (third electrode 17 or neutral electrode 18) on the other hand.

An electrode voltage U_{E} between the first electrode 15 and the third electrode 17 can be referred to as the first electrode voltage U_{E1}, and an electrode voltage U_{E} between the second electrode 16 and the third electrode 17 can be referred to as the second electrode voltage U_{E2}.

In the embodiment illustrated in FIG. 3 supply apparatus 11 comprises a transformer 41 having a primary winding 42 and a secondary winding 43. The secondary winding 43 is electrically connected with one terminal to the first apparatus output 35 and with the terminal at the opposite end with third apparatus output 37. A center tap of secondary winding 43 is in this embodiment electrically connected with the second apparatus output 36. Connected in parallel to primary winding 42 is a capacitor 44. In series with the capacitor 44 and the primary winding 42, a controlled switch 45 is arranged. The transformer 41, the capacitor 44 and the controlled switch 45 form an output stage 46 to which the power supply output 24 is connected. By means of the output stage 46, high voltage impulses can be created that can be provided to instrument 12 or applied to electrodes 15, 16, 17. For this purpose, the controlled switch 45 is controlled by a control device 47 of supply apparatus 11, particularly in order to create and provide high voltage impulses according to a preset operating mode of supply apparatus 11.

In modification to the illustration in FIG. 3, first apparatus output 35 and second apparatus output 36 can also be connected with two individual secondary windings and/or transformers in order to produce the electrode voltages U_{E1} and U_{E2}.

FIG. 4 illustrates a circuit diagram or a block diagram of another embodiment of a supply apparatus 11 as well as an instrument 12. In modification to the embodiment according to FIG. 3, the power supply output 24 is electrically connected with the apparatus output and according to the example first apparatus output 35 and third apparatus output 37 in the supply apparatus 11 according to FIG. 4, so that the output voltage U_{A} applies between first apparatus output 35 and third apparatus output 37. The output current I_{A} flows from supply apparatus 11 to the instrument 12.

In the instrument 12 illustrated in FIG. 4 instrument circuit 29 is more complex than in the embodiment according to FIG. 3. Also, in the instrument 12 according to FIG. 4, first electrode 15 is electrically connected with first instrument input 32 via coupling capacitor 31. The second electrode 16 is electrically connected with third electrode 17 as well as third instrument input 34 via the windings of an autotransformer 51 and a series capacitor 52 connected in series therewith. A center tap of autotransformer 51 between its two windings is electrically connected with first instrument input 32 via a switching device 53. Depending on an operating condition of an operating unit 54, switching device 53 is switched between a conductive state and a blocking state. The operating unit 54 can comprise one or more manually operable push buttons, switches or other operating elements 55 that are accessibly arranged on the instrument 12 and that can be operated or actuated by an operator.

For example, the operating unit 54 is electrically connected with second instrument input 33 that is communicatively connected with control device 47 of supply apparatus 11 via second apparatus output 36, so that the control device 47 can control power supply 23 depending on the operating condition of operating unit 54. In doing so, the output voltage U_{A} can be turned on, turned off or its absolute value or amplitude can be changed, for example.

The supply apparatuses 11 according to FIGS. 3 and 4 can comprise an operating interface 59 by means of which an operator can set an operating mode of supply apparatus 11, which is characterized by an operating mode parameter M (FIG. 5). Optionally, also one or more additional parameters can be preset or adjusted using the operating interface, for example an electrical power, a crest factor, a waveform of the output voltage U_{A}, etc.

In all embodiments, instrument 12 can have an operating unit 54 or can be configured without operating unit. In all embodiments, additionally or alternatively to the operating unit 54 provided on the instrument 12, the operation can be carried out using the operating interface 59 on the supply apparatus 11, which may also include separate hand and/or foot switches, etc., connected to supply apparatus 11 via cable or wirelessly.

The setpoint adjustment device 26 mentioned in the context of the embodiments according to FIGS. 3 and 4 comprises a determination unit 60 as well as a correction unit 61. The determination unit 60 is configured to determine the setpoint output voltage U_{S} depending on the operating mode parameter M (that is depending on the selected operating mode of supply apparatus 11) and a correction value C provided by the correction unit 61. For example, an operating mode voltage U_{M} can be assigned to each operating mode parameter M, which is modified and particularly increased based on the correction value C, whereby a corrected operating mode voltage U_{MC} is obtained. Depending from this corrected operating mode voltage U_{MC}, the setpoint output voltage U_{S} can be determined and provided to the power supply 23.

In a preferred embodiment the operating mode parameter M corresponds to an operating mode voltage U_{M} and the correction value C to a correction voltage U_{C} that are summed up resulting in the corrected operating voltage U_{MC} as illustrated in FIG. 6. A setpoint output unit 62 then sets the setpoint output voltage U_{S} based on the corrected operating mode voltage U_{MC}.

For determining the correction value C or the correction voltage U_{C}, the actual value of the output current I_{A} is transmitted to the correction unit 61. Moreover, the impedance parameter ZI that characterizes the impedance of instrument 12 and particularly instrument circuit 29 is known to correction unit 61. Based on the impedance parameter ZI and the actual value of the output current I_{A}, the correction value C and according to the example the correction voltage U_{C} can be determined according to the following equation:

$${U}_{c} = Z \cdot {I}_{A} ,$$

whereby the impedance Z of the instrument is obtained from the impedance parameter ZI or is equal to the impedance parameter ZI.

Then the output voltage U_{A} is controlled in closed-loop manner according to the setpoint output voltage U_{S} by means of the power supply 23 having the power supply controller 25. For this purpose, the output voltage U_{A} can be detected via voltage sensor 27 and a difference voltage U_{D} between the setpoint output voltage and the actual value of the output voltage U_{A} can be calculated and then provided to the power supply controller 25. The power supply controller 25 controls the output voltage U_{A} in a closed-loop manner with the aim of minimizing the provided difference voltage U_{D}.

By changing and particularly by increasing the corrected operating mode voltage U_{MC} compared to operating mode voltage U_{M} based on the correction value C or the correction voltage U_{C}, reactive voltages can be compensated that occur due to instrument circuit 29 and/or parasitic effects. Thereby a deviation between an electrode voltage U_{E} applied on one of the electrodes 15, 16, 17 of instrument 12 compared to the operating mode voltage U_{M} defined by operating mode parameter M is reduced or eliminated. In the ideal case the electrode voltage U_{E} is equal to the operating mode voltage U_{M}.

For example, correction unit 61 can comprise a computing unit 65 and a memory 66. The computing unit 65 can be a suitable circuit for carrying out mathematical and/or logical operations, for example a microcomputer, ASIC or the like. The memory 66 can be a volatile and/or non-volatile memory. In the memory 66, the impedance parameter ZI can be stored for the instrument 12 or each instrument 12 that can be connected to supply apparatus 11. In this case supply apparatus 11 and instrument 12 can be configured to identify the instrument or the instruments. For example, the instrument 12 can comprise an instrument memory 67 in which an identifier K is stored that can be read out and provided to the correction unit 61 when the electrical connection to the supply apparatus 11 is established. Based on the identifier K, the impedance parameter ZI of the instrument 12 can be determined. A respective assignment in form of a table or the like can be stored in the memory 66.

Alternatively, it is also possible to store and read out the impedance parameter ZI of the instrument 12 in or from instrument memory 67 if the instrument 12 is connected to the supply apparatus 11. Then the impedance parameter ZI is directly available for the correction unit 61. In any case, the correction voltage U_{C} can be determined according to a function and particularly a multiplication of the actual value of the output current I_{A} and the provided or determined impedance parameter ZI. The actual value of the output current I_{A} and the impedance parameter ZI can also be combined with each other using other functions in order to obtain a correction value C, for example a correction factor with which the operating mode voltage U_{M} can be multiplied.

An exemplary temporal progress of an electrode current I_{E} flowing from one of the electrodes 15, 16 into the tissue G to be treated as well as the electrode voltage U_{E} at this electrode is illustrated in FIG. 7. It is apparent that the electrode voltage U_{E} remains constant independent from the absolute value of the electrode current I_{E} during each of the impulses applied to the electrode 15, 16. This effect is due to the fact that the reactive voltage component about which the electrode voltage U_{E} would be reduced in the non-compensated case due to the current, is compensated using the correction value C or the correction voltage U_{C}. The electrode voltage U_{E} applied at the electrode 15, 16 therefore corresponds to the desired operating mode voltage U_{M} that shall in fact be applied to the electrode 15, 16 based on the selected operating mode (defined by the operating mode parameter M).

In an embodiment of the invention the electrode voltage U_{E} can be produced during phases or in one or more time intervals without the voltage compensation explained above. At least one time interval without voltage compensation of the electrode voltage U_{E} can be combined with at least one time interval with voltage compensation of the electrode voltage U_{E} in a temporally indirect or direct sequence. For example, an arbitrary number N of time intervals with voltage compensation of the electrode voltage U_{E} can be followed by an arbitrary number M of time intervals with voltage compensation of the electrode voltage U_{E} or vice versa. Thereby the number N and the number M can be arbitrary natural numbers (1, 2, 3, . . . ). In doing so, optionally additional advantageous effects during treatment of biological tissue can be created.

The invention refers to a supply apparatus 11 and a method for supplying an electromedical instrument 12 with electrical energy. An adjustable and/or selectable operating mode parameter M defines an operating mode voltage U_{M} that indicates a set point value for an electrode voltage U_{E} at an electrode 15, 16 of the electromedical instrument 12. At a power supply output 24 of a power supply 23 of supply apparatus 11 an impressed output voltage U_{A} is provided in accordance with a set point output voltage U_{S} as well as a variable output current I_{A} is provided for the electromedical instrument 12. Due to the impedance of instrument 12, a reactive voltage can be created in the electrical path to the electrode 15, 16, so that the electrode voltage U_{E} does not correspond to the desired operating mode voltage U_{M}. For this reason, the operating mode voltage is corrected based on a correction value C, and particularly a correction voltage U_{C}, and forms a corrected operating mode voltage U_{MC} based on which the set point output voltage U_{S} is provided for the power supply 23. The correction value C or correction voltage U_{C} are determined depending on an actual value of the output current I_{A} as well as an impedance parameter ZI that characterizes the impedance of the instrument 12.

1. A supply apparatus configured for supply of an electromedical instrument with electrical energy, wherein the supply apparatus comprises:
a power supply having a power supply output, the power supply being configured to provide at its power supply output a load-dependent output current (I_{A}) and an impressed output voltage (U_{A}) corresponding to a set point output voltage (U_{S}),
a set point adjustment device comprising a determination unit and a correction unit, wherein the correction unit is configured to determine a correction value (C) depending on a present actual value of the output current (I_{A}) and depending on at least one impedance parameter (ZI) characterizing an impedance of the electromedical instrument (to be supplied, and wherein the determination unit is configured to determine the set point output voltage (U_{S}) based on a set operating mode parameter (M) and the correction value (C).

2. The supply apparatus according to claim 1, wherein the operating mode parameter (M) is an operating mode voltage (U_{M}) and the correction value (C) is a correction voltage (U_{C}) and wherein the determination unit is configured to determine a corrected operating mode voltage (U_{MC}) based on the operating mode voltage (U_{M}) and the correction voltage (U_{C}) and to transmit it to a setpoint output unit that is configured to determine the setpoint output voltage (U_{S}) based on the corrected operating mode voltage (U_{MC}) and provide it to the power supply.

3. The supply apparatus according to claim 2, wherein the determination unit is configured to form the corrected operating mode voltage (U_{MC}) as a sum of the operating mode voltage (U_{M}) and the correction voltage (U_{C}).

4. The supply apparatus according to claim 1, wherein the at least one impedance parameter (ZI) is stored in the correction unit in an unchangeable or changeable manner.

5. The supply apparatus according to claim 1, wherein the supply apparatus is configured to determine a characteristic value (K) of an electromedical instrument connected to the supply apparatus and provide it to the correction unit, which is configured to determine the at least one impedance parameter (ZI) based on the characteristic value (K).

6. The supply apparatus according to claim 5, wherein the characteristic value (K) comprises the at least one impedance parameter (ZI) or is the at least one impedance parameter (ZI).

7. The supply apparatus according to claim 5, wherein the characteristic value (K) describes the electromedical instrument or an instrument type and the correction unit is configured to assign the characteristic value (K) to the at least one impedance parameter (ZI).

8. The supply apparatus according to claim 1, wherein the correction unit is configured to determine the at least one impedance parameter (ZI) depending on an operating mode.

9. The supply apparatus according to claim 2, wherein the correction unit is configured to calculate the correction value (C) and the correction voltage (U_{C}) by means of a preset function.

10. The supply apparatus according to claim 5, wherein the at least one impedance parameter (ZI) depends on a frequency of the output current (I_{A}) and/or the impressed output voltage (U_{A}).

11. A system comprising an electromedical instrument connected to the supply apparatus of claim 1.

12. The system according to claim 11, wherein the electromedical instrument comprises at least one electrode and an instrument circuit with which the electrode or at least one of the electrodes is electrically connected.

13. The system according to claim 12, wherein the instrument circuit comprises a capacitive and/or inductive component.

14. The system according to claim 12, wherein the instrument circuit comprises a coupling capacitor that is electrically connected with the electrode or at least one of the electrodes.

15. A method for supplying an electromedical instrument with electrical energy by a supply apparatus, wherein the method comprises:
providing a load-dependent output current (I_{A}) and an impressed output voltage (U_{A}) on a power supply output of a power supply of the supply apparatus, so that the output voltage (U_{A}) corresponds to a setpoint output voltage (U_{S}),
determining a correction value (C) depending on a present actual value of the output current (I_{A}) and depending on an impedance parameter (ZI) that characterizes an electrical impedance of the electromedical instrument to be supplied, and
determining the setpoint output voltage (U_{S}) based on a set operating mode parameter (M) of the supply apparatus and the correction value (C).
